(19) United States
(12) Patent Application Publication
(10) Pub. No.: US 2016/0038521 A1
(43) Pub. Date: Feb. 11, 2016

(54) THERAPEUTIC METHODS

(71) Applicants: UNIVERSITY OF IOWA RESEARCH FOUNDATION, Iowa City, IA (US); UNIVERSITY OF NEBRASKA MEDICAL CENTER, Omaha, NE (US)

(72) Inventors: Beverly L. Davidson, Iowa City, IA (US); Colleen S. Stein, Iowa City, IA (US); Mark Schultz, Iowa City, IA (US); Luis Tecedor, Iowa City, IA (US); Tammy L. Kielian, Lincoln, NE (US)

(73) Assignee: UNIVERSITY OF IOWA RESEARCH FOUNDATION, Iowa City, IA (US)

(21) Appl. No.: 14/776,558

(22) PCT Filed: Mar. 17, 2014
(86) PCT No.: PCT/US14/30714

§ 371 Date: Sep. 14, 2015

Related U.S. Application Data
(60) Provisional application No. 61/798,361, filed on Mar. 15, 2013.

(51) Int. Cl. A61K 31/704; A61K 31/56

(52) U.S. Cl. CPC A61K 31/704; A61K 31/56

(57) ABSTRACT

Certain embodiments of the present invention provide a method for treating or preventing juvenile neuronal ceroid lipofuscinosis (JNCL) in an animal comprising administering CBX, GRA, or GZA to the animal.

### RELATED APPLICATION

This application claims priority to U.S. Provisional Patent Application No. 61/798,361 filed Mar. 15, 2013, the entirety of which is incorporated herein by reference.

### BACKGROUND OF THE INVENTION

Juvenile neuronal ceroid lipofuscinosis (JNCL) is a progressive neurodegenerative disease that begins in early childhood (reviewed in Jalanko and Braulke, 2009). Symptoms first appear as visual impairment at about 6-7 years of age, and progress rapidly to blindness. Disease proceeds to include seizures and progressive decline in motor and cognitive skills. Death usually occurs in the second decade. JNCL is caused by autosomal recessive inheritance of mutations in the CLN3 gene. The incidence of JNCL is estimated at 1/100,000 globally and as high as 1/20,000 in northern Europe. The mechanism of disease pathogenesis is unknown, and there are no effective therapies. Studies in our lab indicate that functional impairment of brain vascular endothelial cells may be a key element in the pathogenesis of JNCL. A reporter mouse study indicates prominent CLN3 expression in brain endothelium (Eliason et al., 2007), and additional studies indicate that endothelial cells from JNCL mice display multiple cellular defects.

Currently there is a need for agents that are useful for treating or preventing JNCL. JNCL is a progressive neurodegenerative disease with onset in early childhood and shortened lifespan. There are no effective treatments for JNCL.

### SUMMARY OF THE INVENTION

Accordingly the invention provides a method for treating or preventing juvenile neuronal ceroid lipofuscinosis (JNCL) in an animal comprising administering a therapeutic agent that comprises (or consists of) carbenoxolone (CBX) or the related compounds glycyrrhetinic acid (GRA) and/or glycyrrhizic acid (GZA) to the animal. In certain embodiments, the therapeutic agent is CBX. In certain embodiments, the therapeutic agent is administered at a dosage in the range of 0.05 to less than about 50 mg/kg/day. The typical range of oral dose to an adult is 50 to 300 mg/day.

In certain embodiments, the present invention provides CBX, GRA, or GZA for the prophylactic or therapeutic treatment of juvenile neuronal ceroid lipofuscinosis (JNCL).

In certain embodiments, the present invention provides the use of a compound of CBX, GRA, or/and GZA compound to prepare a medicament for treating juvenile neuronal ceroid lipofuscinosis (JNCL) in an animal (e.g., a mammal, such as a human).

### BRIEF DESCRIPTION OF THE FIGURES

FIG. 1 depicts CLN3.

FIGS. 2A and 2B. Carbenoxolone reduces Cdc42 activity in CLN3-null JNCL mouse brain endothelial cells (MBEC). A) Cdc42 activity levels are significantly elevated in CLN3-null (CLN3−/−) MBEC, compared to CLN3-expressing (CLN3-R) MBEC. B) Carbenoxolone (CBX) treatment (50 μM for 2 h) does not alter Cdc42 activity level in CLN3-R MBEC (left panel), but significantly reduces Cdc42 activity in CLN3−/− MBEC (right panel). *p<0.05.

FIG. 3: CBX corrects Cdc42-GTP levels. Cdc42-GTP levels after CBX 25 μM for 2 hours or saline (mock) treatment. Data represent the mean of three independent experiments, error bars±SEM (1-way ANOVA with TUKEY post-hoc, (*, p<0.05, n.s.=not significant).

FIG. 4. Carbenoxolone normalizes migration of CLN3-null JNCL mouse brain endothelial cells. Cell migration induced by a scratch in cell monolayers was measured using live cell microscopy and quantified by T-Scratch software. CLN3-expressing (CLN3-R) and CLN3-null (CLN3−/−) MBEC were grown to confluence. A gap in the monolayer was made by “scatch wound”, and the rate of cell migration to fill in the gap was monitored by live cell microscopy. CLN3−/− MBEC show a delayed ability to fill in the gap. Carbenoxolone treatment corrects this defect. Cells were either treated with CBX or left untreated (mock) in the presence or absence of CBX. Data are collected overnight. Data represent the mean of three independent experiments, error bars±SEM (2-way ANOVA with Bonferroni post-hoc correction, *, p<0.05).

FIG. 5. Correction of fluid-phase endocytosis by CBX. Hoechst 33342 to label DNA (blue) and saline (mock) or 25 μM CBX was added to cell culture media for 30 minutes. Fluid-phase endocytic uptake in the presence of CBX or PBS was determined by uptake of fluorescent dextran (green). Extracellular dextran was quenched by Red-40 and cells were imaged by epifluorescence. ImageJ was used to calculate fluorescence intensity.

Data represent the mean of three independent experiments, error bars±SEM (1-way ANOVA with TUKEY post-hoc, ***, p<0.0001). Scale bar=10 μm.

FIG. 6. Carbenoxolone restores caveolin-1 transport to the cell membrane in CLN3-null JNCL mouse brain endothelial cells. CLN3-expressing (CLN3-R) and CLN3-null (CLN3−/−) MBEC were untreated or cultured with 25 μM carbenoxolone for 2 h, then immunofluorescently stained for caveolin-1. Without treatment, caveolin-1 transport to the plasma membrane is impaired in CLN3−/− MBEC. Carbenoxolone restores normal caveolin-1 trafficking to the plasma membrane.

FIG. 7. Carbenoxolone restores normal fluidity to the cell membrane in CLN3-null JNCL mouse brain endothelial cells. CLN3-expressing (CLN3-R) and CLN3-null (CLN3−/−) MBEC were untreated or treated with carbenoxolone (25 μM) for 2 h. Apical cell membranes were then labeled with Alexa-488-cholera toxin subunit B (A488-CTB), and the fluidity of lipid microdomains were assessed by fluorescence recovery after photobleaching (FRAP). For FRAP, a region was photobleached and the rate of fluorescence recovery as a consequence of A488-CTB diffusion was measured. In the absence of carbenoxolone, CLN3−/− MBEC showed faster recovery, indicating higher membrane fluidity. Carbenoxolone treatment imparted stability to CLN3−/− MBEC membranes, reducing the recovery rate to control MBEC level. Images were taken for 250 seconds and Zen software used to analyze recovery of fluorescence. Representative data from three independent experiments with at least 15 cells per group are shown. CBX 25 μM for 2 hours or mock treatment. Error bars±SEM (2-way ANOVA with Bonferroni post-hoc correction, *, p<0.05).

FIG. 8: CBX corrects cholesterol distribution at the plasma membrane. Plasma membranes are detergent-free fractionated and cholesterol levels in each fraction are quantified by the Amplex Red® Cholesterol assay kit. A representative experiment from four independent experiments is shown with CBX 25 μM for 2 hours or mock treatment.

FIG. 9: Model depicting how Hoechst enters the brain during hypotonicity. (Top) Schematic representing the mechanism by which Hoechst gains entry into the brain parenchyma during hypotonic treatment. (Bottom) Hoechst penetration in Cln3^{+/-} mice exposed to isotonic or hypotonic treatment. Wheat germ aggluttinin (WGA) (green) labels endothelial cells and Hoechst (blue) signal outside the WGA is evident in brain parenchyma. Scale bar=100 μm.

FIG. 10: CBX corrects dye permeability in vivo. CLN3⁺ or Cln3^{-/-} mice were gavaged daily with saline or 20 mg/kg of CBX for two weeks. 24 hrs after the last treatment, mice were perfused via the left heart ventricle with WGA (green), a hypotonic solution containing Hoechst (blue) (Invitrogen), saline, and fixed with PFA. Brain slices (50 μm) were imaged by confocal microscopy. Data are representative of 5 mice per group. Scale bar=50 μm.

FIG. 11: CBX treatment reduces Cln3^{-/-} autofluorescent inclusions. Thin (50 μm) brain sections from mice treated as in were imaged under low magnification in the red channel to detect autofluorescence. Four images were taken of each cingulate cortex, and fluorescence intensity averaged in ImageJ. Data represent a minimum of 4 mice per group. Error bars±SEM (1-way ANOVA with TUKEY post-hoc, *, p<0.05) Scale bar=200 μm.

### DETAILED DESCRIPTION

In certain embodiments, the present invention provides a systemic administration of CBX, GRA, or GZA to treat JNCL and/or reduce the symptoms of JNCL in patients. As a systemic application, CBX, GRA, or GZA accesses the brain vascular endothelial cells, with potential to improve endothelial cell function and indirectly improve neuronal health, reducing JNCL symptoms.

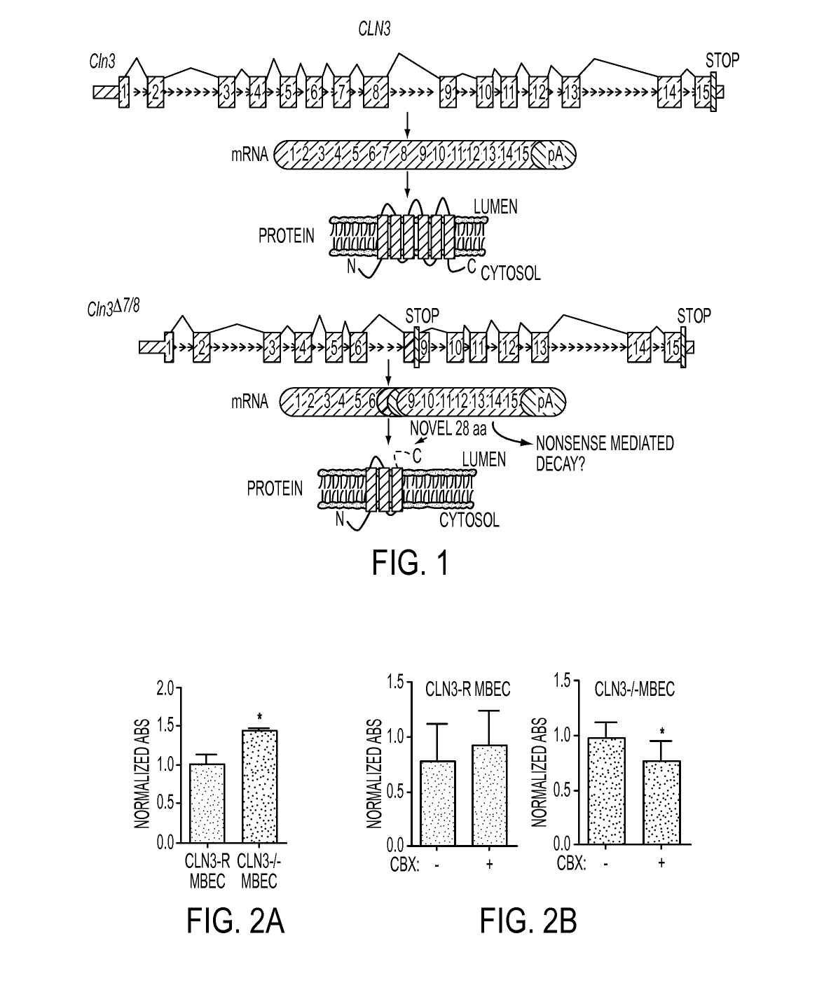

In vitro studies by the inventors indicate that CBX treatment of brain endothelial cells derived from a mouse model of juvenile neuronal ceroid lipofuscinosis (JNCL) alleviates cellular dysfunctions and corrects endothelial cell defects. The inventors have determined that addition of CBX to culture media corrects cellular phenotypes (FIGS. 1-5). Systemic administration of CBX would likely correct endothelial defects in vivo as the endothelium is exposed to the drug, which in-turn would improve general central nervous system (CNS) health and neuronal function, and prevents or lessens JNCL symptoms. GRA and GZA, which are related to CBX and have reported similar effects (Juszczak and Swiergiel, 2009), may have similar therapeutic effects. No other research groups are known to have tested or plan to test systemic administration of CBX, GRA, or GZA for the treatment of JNCL. While CBX, GRA, or GZA have been used clinically or experimentally in humans, their systemic application for treatment of JNCL is a novel concept.

CBX is a synthetic derivative of GZA, which in turn is derived from GRA, a natural component of licorice root. CBX, GRA, and GZA have broad effects in vitro and in vivo. The molecular mechanisms are not fully understood, and may involve described abilities to inhibit 11-beta-hyroxysteroid dehydrogenase, an enzyme involved in glucocorticoid synthesis, or to block hemi-channels or gap junction communication between cells (reviewed in Juszczak and Swiergiel, 2009). CBX was originally found to be useful in humans for healing peptic ulcers (Guslandi et al., 1980), and more recently has been found beneficial for treating insulin insensitive diabetes (Andrews et al., 2003). It has been cited to have neuroprotective effects in models of traumatic brain injury, stroke, and epilepsy (Frantseva et al., 2002b; Frantseva et al., 2002a; Gareri et al., 2004; Khorasani et al., 2009; Vakili et al., 2009; Connors, 2012). Since CBX has little or no ability to pass the blood-brain barrier (BBB) (Leshchenko et al., 2006), neuroprotective effects of systemic CBX in these disorders may rely on focal loss of BBB. With respect to the present invention of treating JNCL with systemic administration of CBX, GRA, or GZA, the drug accesses vascular endothelial cells in the CNS via the circulation for potential therapeutic effect.

CBX, GRA, or GZA can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula Ito the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.05 to about 50 mg/kg per day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention will now be illustrated by the following non-limiting Example.

### EXAMPLE 1

Individuals with JNCL exhibit mental decline, with symptoms being observed 2-4 years after visual onset of progressive seizures. Patients exhibit rapid cognitive, behavioral, and physical decline, and death usually occurs by second decade of life. 85% of patients have a deletion in the CLN3 gene (FIG. 1). A β-gal knock in reporter was generated by inserting β-Gal pA in between region 1 and region 8 (CLN3-null (Cln3^{-/-})). Cln3^{-/-} mouse exhibits neurological defects, reduced seizure threshold, modified motor phenotypes, and reduced activity. The function of CLN3 is unclear; however, Cln3 regulates Cdc42, and loss of this regulation reduces fluid-phase endocytosis.

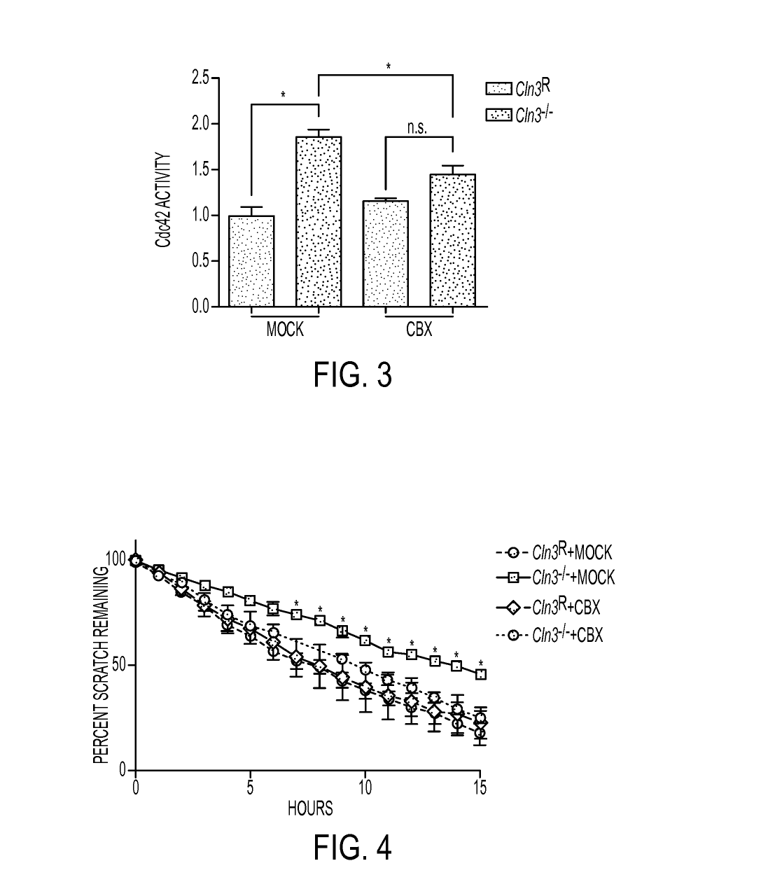

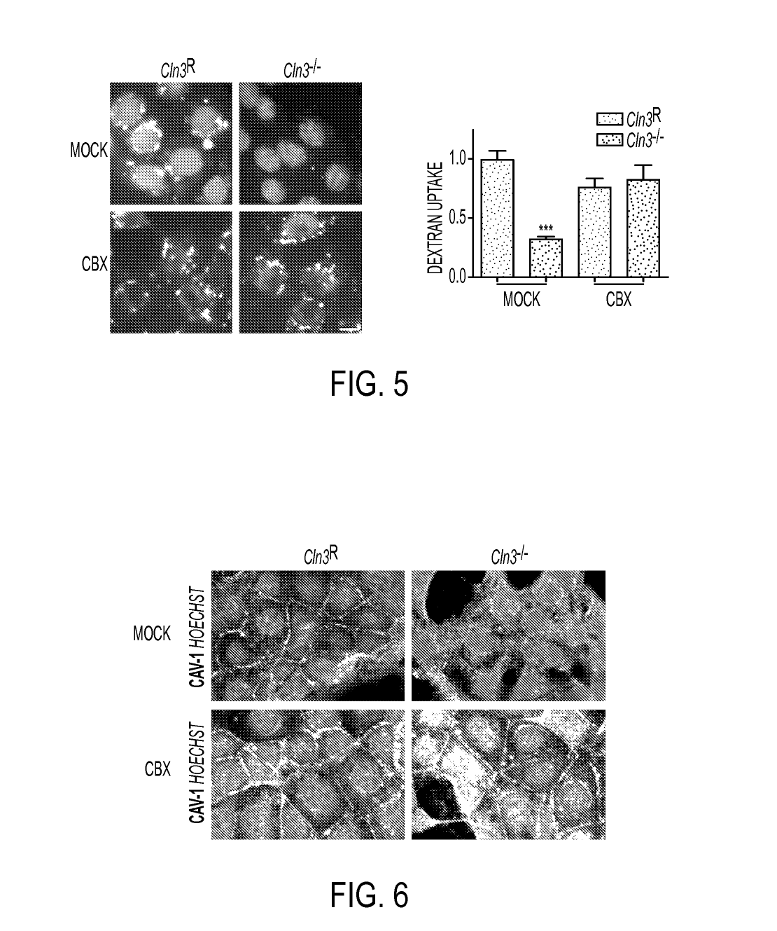

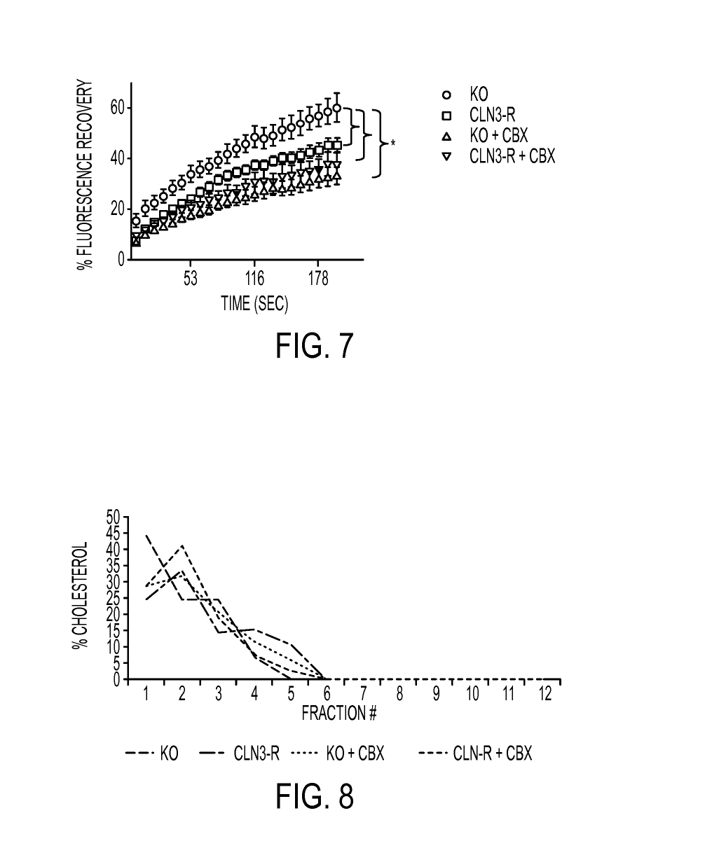

Cells derived from JNCL patients or mouse models show defects along various pathways. Recent studies by the inventors indicate that brain endothelial cells from JNCL mice have defects in endocytosis, cell migration, cell surface expression of caveolin-1, and cell membrane fluidity. Several of these processes are known to be regulated by Cdc42, a small intracellular molecule that switches between active and inactive forms. It was found that the level of active Cdc42 is abnormally elevated in JNCL endothelial cells. One report in the literature showed that gap junction inhibitors such as CBX reduced Cdc42 activity (Liu et al., 2010). Also, a connectivity map (www.broadinstitute.org/cmap) query by the inventors showed that CBX ranked highly (amongst 1309 different compounds) as inducing gene expression changes inversely correlated with JNCL. When the inventors treated JNCL endothelial cells with CBX, they discovered that it effectively normalized Cdc42 activity (FIGS. 2A and 2B). CBX reduces Cdc42-GTP in Cln3⁴⁻⁻ MBECs (FIG. 3), and CBX CLN3⁻⁻ increases cell migration (FIG. 4). Short CBX treatment increases fluid-phase endocytosis in Cln3^{-/-} MBECs. CBX corrected in vitro Cln3^{-/-} MBECs defects and were Cdc42 and cav-1 dependent. Moreover, they found that CBX treatment also corrected the other cellular defects observed in JNCL endothelial cells (FIGS. 5-7). CBX also corrects cholesterol distribution at the plasma membrane (FIG. 8).

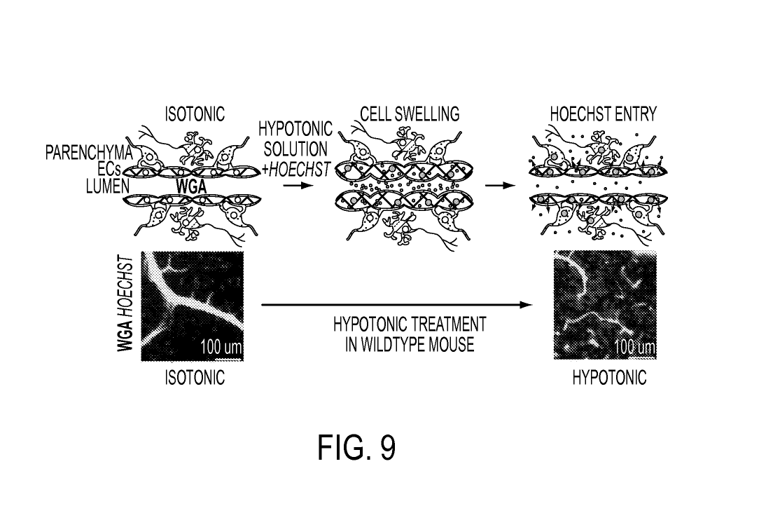

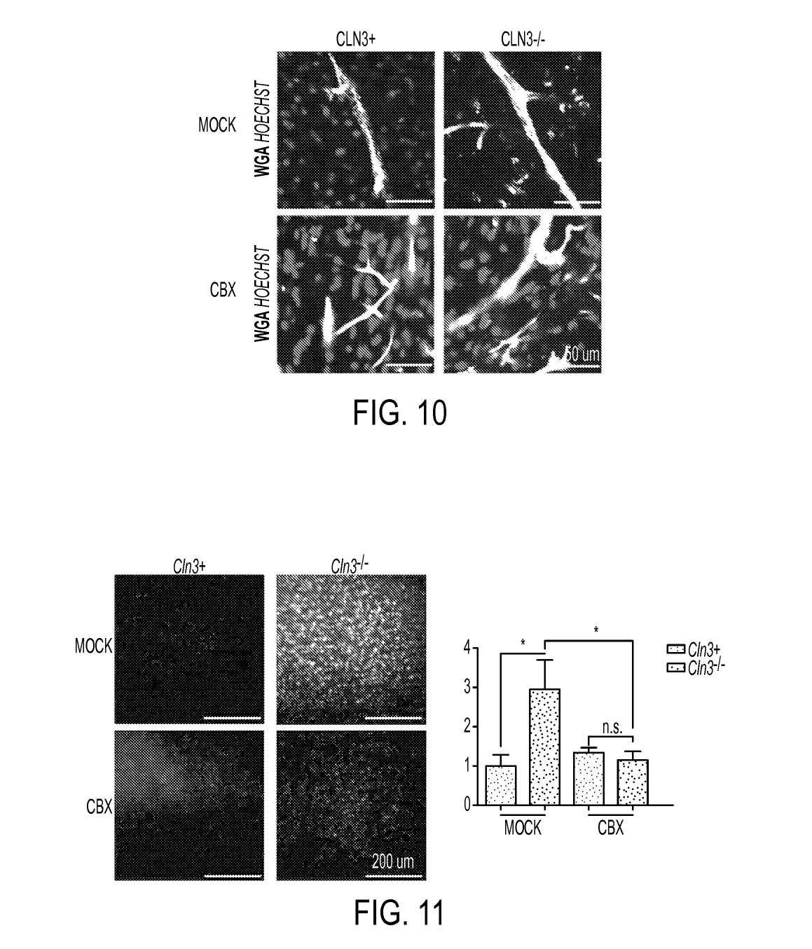

The inventors also prepared an in vivo model depicting how Hoechst enters the brain during hypotonicity (FIG. 9), and how CBX corrects dye permeability in vivo (FIG. 10). They also observed that CBX treatment reduces Cln3^{-/-} autofluorescent inclusions (FIG. 11).

### REFERENCES

Andrews R C, Rooyackers O, Walker B R (2003) Effects of the 11 beta-hydroxysteroid dehydrogenase inhibitor carbenoxolone on insulin sensitivity in men with type 2 diabetes. J Clin Endocrinol Metab 88:285-291.
Connors B W (2012) Tales of a dirty drug: carbenoxolone, gap junctions, and seizures. Epilepsy Curr 12:66-68.
Eliason S L, Stein C S, Mao Q, Tecedor L, Ding S L, Gaines D M, Davidson B L (2007) A knock-in Reporter model of Batten disease. J Neurosci 27:9826-9834.
Frantseva M V, Kokarovtseva L, Perez Velazquez J L (2002a) Ischemia-induced brain damage depends on specific gap junctional coupling. J Cereb Blood Flow Metab 22:453-462.
Frantseva M V, Kokarovtseva L, Naus C G, Carlen P L, MacFabe D, Perez Velazquez J L (2002b) Specific gap junctions enhance the neuronal vulnerability to brain traumatic injury. J Neurosci 22:644-653.
Gareri P, Condorelli D, Belluardo N, Russo E, Loiacono A, Barresi V, Trovato-Salinaro A, Mirone M B, Ferreri Ibbadu G, De Sarro G (2004) Anticonvulsant effects of carbenoxolone in genetically epilepsy prone rats (GEPRs). Neuropharmacology 47:1205-1216.
Guslandi M, Cambielli M, Tittobello A (1980) Carbenoxolone maintenance in cimetidine-healed patients. Scand J Gastroenterol 15:369-371.
Jalanko A, Braulke T (2009) Neuronal ceroid lipofuscinoses. Biochim Biophys Acta 1793:697-709.
Juszczak G R, Swiergiel A H (2009) Properties of gap junction blockers and their behavioural, cognitive and electrophysiological effects: animal and human studies. Prog Neuropsychopharmacol Biol Psychiatry 33:181-198.
Khorasani M Z, Hosseinzadeh S A, Vakili A (2009) Effect of central microinjection of carbenoxolone in an experimental model of focal cerebral ischemia. Pak J Pharm Sci 22:349-354.
Leshchenko Y, Likhodii S, Yue W, Burnham W M, Perez Velazquez J L (2006) Carbenoxolone does not cross the blood brain barrier: an HPLC study. BMC Neurosci 7:3.
Liu X, Hashimoto-Torii K, Torii M, Ding C, Rakic P (2010) Gap junctions/hemichannels modulate interkinetic nuclear migration in the forebrain precursors. J Neurosci 30:4197-4209.
Vakili A, Hosseinzadeh S A, Khorasani M Z (2009) Peripheral administration of carbenoxolone reduces ischemic reperfusion injury in transient model of cerebral ischemia. J Stroke Cerebrovasc Dis 18:81-85.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for treating or preventing juvenile neuronal ceroid lipofuscinosis (JNCL) in an animal comprising administering a therapeutic agent to the animal, wherein the therapeutic agent is carbenoxolone (CBX), glycyrrhetinic acid (GRA) and/or glycyrrhizic acid (GZA).

2. The method of claim 1, wherein the therapeutic agent is CBX.

3. The method of claim 1, wherein the therapeutic agent is administered at a dosage in the range of about 0.05 mg/kg/day to about 50 mg/kg/day.

4. The method of claim 1, wherein the therapeutic agent is administered orally or parenterally.

5. (canceled)

6. (canceled)

7. The method of claim 1, wherein the animal is a mammal.

8. The method of claim 7, wherein the mammal is a human.

9. The method of claim 2, wherein the therapeutic agent is administered at a dosage in the range of about 0.05 mg/kg/day to about 50 mg/kg/day.

10. The method of claim 2, wherein the therapeutic agent is administered orally or parenterally.

11. The method of claim 2, wherein the animal is a human.

12. The method of claim 1, wherein the therapeutic agent is GRA.

13. The method of claim 12, wherein the therapeutic agent is administered at a dosage in the range of about 0.05 mg/kg/day to about 50 mg/kg/day.

14. The method of claim 12, wherein the therapeutic agent is administered orally.

15. The method of claim 12, wherein the animal is a human.

16. The method of claim 1, wherein the therapeutic agent is GZA.

17. The method of claim 16, wherein the therapeutic agent is administered at a dosage in the range of about 0.05 mg/kg/day to about 50 mg/kg/day.

18. The method of claim 16, wherein the therapeutic agent is administered orally or parenterally.

19. The method of claim 16, wherein the animal is a human.
